(19) United States
(12) Patent Application Publication
(10) Pub. No.: US 2020/0291350 A1
(43) Pub. Date: Sep. 17, 2020

(54) HEPATOCYTE CONSTRUCT AND METHOD FOR PRODUCING THE SAME

(71) Applicants: NIHON KOHDEN CORPORATION, Tokyo (JP); TOKYO WOMEN'S MEDICAL UNIVERSITY, Tokyo (JP)

(72) Inventors: Botao GAO, Tokyo (JP); Yuki KAGAWA, Tokorozawa-shi (JP); Tetsuya OGAWA, Tokorozawa-shi (JP); Hirotsugu KUBO, Tokorozawa-shi (JP); Tatsuya SHIMIZU, Tokyo (JP); Katsuhisa SAKAGUCHI, Tokyo (JP)

(73) Assignees: NIHON KOHDEN CORPORATION, Tokyo (JP); TOKYO WOMEN'S MEDICAL UNIVERSITY, Tokyo (JP)

(21) Appl. No.: 16/502,232

(22) Filed: Jul. 3, 2019

(30) Foreign Application Priority Data
Mar. 12, 2019 (JP) 2019-044725

(51) Int. Cl. C12N 5/071; C12N 5/077; C12N 5/0775

(52) U.S. Cl. CPC C12N 5/067; C12N 5/0656; C12N 2502/1323; C12N 2502/1352; C12N 5/0662

(57) ABSTRACT

The presently disclosed subject matter is to provide a hepatocyte structure, particularly, a hepatocyte structure that mimics nonalcoholic steatohepatitis (NASH).

A hepatocyte construct including an aggregate containing hepatocytes and adherent cells that are non-hepatocytes, and wherein the hepatocytes include ballooned hepatocytes is provided. Further, a method for producing a hepatocyte construct, comprising: (i) forming an aggregate by aggregating a cell group comprising hepatocytes and adherent cells that are non-hepatocytes; and (ii) culturing the aggregate is provided.

### CROSS-REFERENCE TO RELATED APPLICATIONS

This application claims priority from Japanese Patent Application No. 2019-044725 filed on Mar. 12, 2019, the entire contents of which are incorporated herein by reference.

The presently disclosed subject matter relates to a hepatocyte construct and a method for producing a hepatocyte construct.

The liver is known as an important organ that performs gluconeogenesis, glycogen storage, lipid metabolism, production of plasma proteins, and metabolic functions such as bilirubin metabolism, hormone metabolism, vitamin metabolism, drug metabolism, and alcohol metabolism.

In Japan, the number of patients who suffer from nonalcoholic steatohepatitis (NASH) is increasing due to westernization of lifestyle. NASH is a serious disease that progresses into cirrhosis or liver cancer, however, at present, a specific therapeutic method that is effective to NASH has not been found out. Therefore, studies for developing a method or an agent for drastically treating NASH have been conducted throughout the world.

In order to develop a novel therapeutic method for NASH, “a system that mimics NASH on a culture dish” which is useful for evaluating drug efficacy is needed, and the development thereof has also been performed. Several techniques for producing hepatocytes having some of the characteristics observed in cells obtained by biopsy from a patient with NASH are known, and for example, Non-Patent Document 1 discloses a co-culture system using a micropatterning technique. Further, Non-Patent Document 2 discloses a three-dimensional perfusion cell culture system. Further, Non-Patent Document 3 discloses a human liver system that mimics a physiological blood flow condition.

However, human hepatocytes cultured by these techniques can reproduce only “some” of the characteristics (for example, deposition of lipid droplets, infiltration of inflammatory cells, hepatocellular ballooning, appearance of Mallory-Denk bodies, fibril formation around hepatocytes, etc.) of hepatocytes obtained by biopsy from a patient with a liver disease such as NASH, and therefore can hardly be considered as a sufficient NASH model. Further, in order to carry out these techniques, a special processing technique, instrument, or device, or the like is needed, and therefore, there was a problem that the system becomes complicated and the cost increases. In addition, an operational procedure is also complicated, and there was also a problem with reproducibility.

### CITATION LIST

[Non-Patent Document 1] Davidson M. D., et al., Microengineered cultures containing human hepatic stellate cells and hepatocytes for drug development. Integr. Biol. (Camb). 2017 Aug. 14; 9(8): 662-677
[Non-Patent Document 2] Kostrzewski T., et al., Three-dimensional perfused human in vitro model of non-alcoholic fatty liver disease. World J. Gastroenterol. 2017 Jan. 14; 23(2): 204-215
[Non-Patent Document 3] Feaver R. E., et al., Development of an in vitro human liver system for interrogating nonalcoholic steatohepatitis. JCI Insight. 2016 Dec. 8; 1(20): e90954

### SUMMARY

The presently disclosed subject matter is to provide a liver disease model, particularly, a hepatocyte construct that mimics nonalcoholic steatohepatitis (NASH).

The present inventors performed research and development by adding considerations from various aspects. As a result, they surprisingly found that a hepatocyte construct that mimics nonalcoholic steatohepatitis (NASH) is obtained by releasing a sheet-like cell aggregate containing hepatocytes and primate fibroblasts, thereby obtaining a contracted cell sheet, and then culturing the contracted cell sheet. That is, the presently disclosed subject matter includes the following contents.

[1] A hepatocyte construct comprising an aggregate containing hepatocytes and adherent cells that are non-hepatocytes, wherein the hepatocytes include ballooned hepatocytes.
[2] The hepatocyte construct according to [1], wherein the adherent cells are fibroblasts or mesenchymal stem cells.
[3] The hepatocyte construct according to [1] or [2], wherein the adherent cells are primate fibroblasts.
[4] The hepatocyte construct according to any one of [1] to [3], wherein the adherent cells are primate fibroblasts derived from a dermis.
[5] The hepatocyte construct according to any one of [1] to [4], wherein the aggregate is a cell sheet.
[6] The hepatocyte construct according to any one of [1] to [5], wherein sonic hedgehog (SHH) protein expression per cell is higher than that in normal hepatocytes.
[7] The hepatocyte construct according to any one of [1] to [6], wherein the hepatocytes contain a Mallory-Denk body.
[8] The hepatocyte construct according to any one of [1] to [7], wherein the hepatocytes are primary hepatocytes.
[9] The hepatocyte construct according to any one of [1] to [8], wherein the hepatocyte construct expresses α-SMA.
[10] The hepatocyte construct according to any one of [1] to [9], wherein a ratio of the hepatocytes to the adherent cells is from 10:1 to 1:10.
[11] The hepatocyte construct according to any one of [1] to [10], wherein a ratio of the hepatocytes to the adherent cells is from 1:1 to 1:10.
[12] A method for producing a hepatocyte construct comprising (i) forming an aggregate by aggregating a cell group containing hepatocytes and adherent cells that are non-hepatocytes and (ii) culturing the aggregate.
[13] The method according to [12], wherein the adherent cells are fibroblasts or mesenchymal stem cells.
[14] The method according to [12] or [13], wherein the adherent cells are primate fibroblasts.
[15] The method according to any one of [12] to [14], wherein the hepatocytes are primary hepatocytes.
[16] The method according to any one of [12] to [15], wherein the step (i) comprises (i-1) seeding and culturing the hepatocytes on a first culture substrate, (i-2) seeding and culturing the adherent cells on the first culture substrate on which the hepatocytes are seeded and (i-3) forming the aggregate by releasing the cell group containing the hepatocytes and the adherent cells from the first culture substrate.
[17] The method according to any one of [12] to [16], wherein the step (ii) is having the aggregate adhered onto a second culture substrate and culturing the aggregate.
[18] The method according to any one of [12] to [17], wherein in the step (i), a ratio of the hepatocytes to the adherent cells is from 10:1 to 1:10.
[19] The method according to any one of [12] to [18], wherein a ratio of the hepatocytes to the adherent cells is from 1:1 to 1:10.
[20] The method according to any one of [12] to [19], wherein a culture medium used in the method contains glucose at 1 mM to 100 mM.
[21] The method according to any one of [12] to [20], wherein a culture medium used in the method contains insulin at 0.1 μM to 10 μM.
[22] The method according to [16], wherein the first culture substrate is a stimulus-responsive culture substrate.
[23] The method according to [16], wherein the first culture substrate is a temperature-responsive culture substrate.
[24] The method according to [16], [22] or [23], wherein the first culture substrate is coated with a cell-adhesive hydrogel.
[25] A hepatocyte construct which is produced by the method according to any one of [12] to [24].

### DETAILED DESCRIPTION

According to the presently disclosed subject matter, it can be provided a hepatocyte construct that mimics the characteristics of hepatocytes in a liver disease, particularly, nonalcoholic steatohepatitis (NASH) without needing a special processing technique, instrument, or device, or the like unlike the related art.

### BRIEF DESCRIPTION OF THE DRAWINGS

FIG. 1 shows a schematic representation of a strategy to produce hepatocyte constructs (primary human hepatocyte/fibroblast co-culture cell sheets).

FIGS. 2A to 2C show morphologies of a contracted cell sheet obtained by releasing a monolayer of confluent cells from the surface of a stimulus-responsive culture dish. FIG. 2A shows the time-lapse imaging of release and contraction of the primary human hepatocyte (PPH)/normal human dermal fibroblasts (NHDF) cell sheet. FIG. 2B shows photographs of a PHH/NHDF cell sheet (left side) and a PHH/3T3-J2 cell sheet (right side). FIG. 2C shows a graph of area of cell sheets. All data are expressed as mean±SD for at least three values, **p<0.01.

FIGS. 3A and 3B show morphologies of PHH/NHDF cell sheets and PHH/3T3-J2 cell sheets. FIG. 3A shows a phase-contrast micrographs of PHH/NHDF cell sheets and PHH/3T3-J2 cell sheets after one day and four days culture. FIG. 3B shows Hematoxylin and Eosin (H&E)-stained images of PHH/NHDF cell sheets and PHH/3T3-J2 cell sheets on Day 1, 4 and 11. Pronounced cellular enlargement of hepatocytes with pale staining in the cytoplasm (circled cells) was observed in PHH/NHDF cell sheets on day 4 and day 11. Hepatocytes in PHH/3T3-J2 sheets were normal. The bars represent 100 μm.

FIGS. 4A and 4B show the result of quantification of cellular enlargement of hepatocytes in PHH/NHDF cell sheets. In FIG. 4A, immunofluorescence-stained sections for E-Cadherin/DAPI-stained images showed clearly the border of hepatocytes. Green:E-Cadherin, Blue: DAPI (nucleus). The bars represent 100 μm. In FIG. 4B, measurement of cross-section area of each PHH in cell sheets was performed in three independent experiments by Image J software. In each experiment, more than 25 cells were measured. **p<0.01.

FIGS. 5A to 5C show that enlarged hepatocytes in PHH/NHDF co-culture cell sheet were ballooned hepatocytes (day 11). In FIG. 5A, the immunohistochemistry(IHC)-stained sections of PHH/NHDF cell sheets for CK 8/18 showed enlarged hepatocytes which were loss of the brown cytoplasmic keratin staining, but the IHC-stained sections of PHH/3T3-J2 cell sheets showed normal-appearing hepatocytes with homogeneous, brown cytoplasmic keratin staining. The bars represent 100 μm. In FIG. 5B, the immunofluorescence staining of CK 8/18 revealed that irregularly shaped CK 8/18 positive cytoplasmic inclusions located in proximity to the nucleus in the enlarged hepatocytes, which supported the presence of Mallory-Denk bodies (arrow heads). The bars represent 20 μM. In FIG. 5C, the abundant accumulation of lipid droplets was detected in the PHH/NHDF cell sheet after staining with Nile red, while a limited amount of lipid was visible in the PHH/3T3-J2 cell sheet. The bars represent 100 μm.

FIGS. 6A and 6B show the increased secretion of SHH and myofibroblast activation in PHH/NHDF co-culture cell sheets. In FIG. 6A, the measurement of SHH ligands was performed in three independent experiments using two lots of PHHs (Hu8200_A×2+Hu1652×1). In each experiment, more than three samples were measured. Significant increases of SHH production in the PHH/NHDF cell sheet compared with the PHH/3T3-J2 cell sheet on day 4 were found. **p<0.01, *p<0.05. In FIG. 6B, the immunofluorescence-stained sections for α-SMA (red) showed α-SMA positive NHDFs were around ballooned hepatocytes, which indicated myofibroblast activation. The bars represent 100 μm.

FIGS. 7A to 7C show the liver-specific functions of PHH/NHDF cell sheets and PHH/3T3-J2 cell sheets. The measurement of albumin as shown in FIG. 7A and urea as shown in FIG. 7B was performed in three independent experiments using one lot of PHHs (Hu8200A). In each experiment, more than three replicate samples were measured. In FIG. 7C, CYP enzyme activities including CYP1A2 and CYP3A4 were significant lower in PHH/NHDF cell sheets than that in PHH/3T3-J2 cell sheet. The measurement of CYP activities was performed in three independent experiments using two lots of PHHs (Hu8200_A×2+Hu1652×1). In each experiment, more than three samples were measured. **p<0.01, *p<0.05.

FIGS. 8A and 8B show that a co-culture ratio of PHHs and NHDFs modulated the degree of hepatocellular damages and ballooning degeneration. In FIG. 8A, relative to 4:1 co-culture ratio of PHHs and NHDFs, SHH production increased 3.5 times in 1:4 co-culture and 1.6 times in 1:2 co-culture. All data were expressed as mean±SD for more than three values; **p<0.01. In FIG. 8B, the size of hepatocytes was much smaller in 4:1 co-culture ratio of PHHs and NHDFs than that in 1:2 and 1:4 co-culture. The bars represent 100 μm.

(a) to (d) of FIG. 9 show morphologies of PHH/adipose-derived mesenchymal stem cell (ADSC) sheets and PHH/NHDF cell sheets. (a) of FIG. 9 shows a phase-contrast micrograph of a PHH/adipose-derived mesenchymal stem cell (ADSC) sheet (on day 28 after release). (b) of FIG. 9 shows an enlarged view of (a). (c) of FIG. 9 shows a phase-contrast micrograph of a PHH/NHDF cell sheet (on day 28 after release). (d) of FIG. 9 shows an enlarged view of (c). In all, accumulation of lipid droplets was confirmed.

FIGS. 10A and 10B show results of examining the effect of a treatment with mitomycin C on hepatocellular ballooning. A hematoxylin-eosin (H&E)-stained image of a mouse hepatocyte/NHDF cell sheet (day 8) produced using NHDFs treated with mitomycin C (6 μg/mL) for 2.5 hours in FIG. 10A or untreated in FIG. 10B is shown. By the treatment with mitomycin C, hepatocellular ballooning was suppressed. The bars represent 100 μm.

FIGS. 11A and 11B show results of examining the effect of obeticholic acid (OCA) or metformin on ballooning of mouse hepatocytes. A hematoxylin-eosin (H&E)-stained image of a mouse hepatocyte/NHDF cell sheet (day 8) in which obeticholic acid (10 μM) in FIG. 11A or metformin (500 μM) in FIG. 11B was added on day 1 was shown. By adding obeticholic acid (OCA) or metformin, hepatocellular ballooning was suppressed. The bars represent 100 μm.

FIGS. 12A and 12B show results of examining the effect of obeticholic acid (OCA) or metformin on ballooning of human hepatocytes. A hematoxylin-eosin (H&E)-stained image of a PHH/NHDF cell sheet on day 11 or day 4 in which obeticholic acid (10 μM) in FIG. 12A or metformin (500 μM) in FIG. 12B was added on day 1 was shown. By adding obeticholic acid (OCA) or metformin, hepatocellular ballooning was suppressed. The bars represent 100 μm.

FIGS. 13A and 13B show results of examining the effect of a glucose concentration and an insulin concentration on ballooning of human hepatocytes. A hematoxylin-eosin (H&E)-stained image of a PHH/NHDF cell sheet (day 4) cultured at low glucose concentration (5.6 mM)+low insulin concentration (1 nM) in FIG. 13A or at high glucose concentration (25 mM)+high insulin concentration (1 μM) in FIG. 13B was shown. By the culture in a culture medium at low glucose concentration (5.6 mM)+low insulin concentration (1 nM), hepatocellular ballooning was suppressed to some extent. The bars represent 100 μm.

### EMBODIMENTS FOR CARRYING OUT THE DISCLOSED SUBJECT MATTER

Hereinafter, the presently disclosed subject matter will be described through embodiments of the disclosed subject matter, however, the following embodiments do not limit the disclosed subject matter according to the claims, and not all combinations of features described in the embodiments are essential for means for solution of the disclosed subject matter. The matters described in the following “hepatocyte construct”, “method for producing a hepatocyte construct”, and “method for evaluating a factor for treating or preventing a liver disease” can be mutually applied.

### <<Hepatocyte Construct>>

The presently disclosed subject matter provides a hepatocyte construct containing an aggregate containing hepatocytes and adherent cells that are non-hepatocytes, and the hepatocytes include ballooned hepatocytes. In addition, the presently disclosed subject matter provides a hepatocyte construct obtained by the below-mentioned method for producing a hepatocyte construct.

In this description, the “aggregate” refers to a cell group comprising hepatocytes and adherent cells that are non-hepatocytes and in which the cell density per unit volume is higher than that in a cell group seeded on a culture substrate. In one embodiment, the aggregate according to the presently disclosed subject matter can be a contracted cell group (for example, a cell sheet) formed by seeding cells on a culture substrate, releasing a cell group that became confluent or subconfluent, followed by contraction due to the contraction action of the cell group. Further, in another embodiment, the aggregate according to the presently disclosed subject matter may be a cell aggregate obtained by seeding cells on a substrate to which cells are not adhered (for example, a Petri dish or the like), and culturing the cells while allowing a centrifugal force to act toward the bottom face of the substrate (see, for example, Japanese Patent No. 5407343).

In one embodiment, the aggregate included in the presently disclosed subject matter may be “a contracted cell sheet”. In this description, the “contracted cell sheet” refers to a cell sheet obtained by releasing a cell group seeded on a stimulus-responsive culture substrate from the stimulus-responsive culture substrate, followed by spontaneous contraction of the cell group. In one embodiment, the “contracted cell sheet” included in the presently disclosed subject matter may be a sheet contracted to an area that is ¼ or less, more preferably ⅕ or less, further more preferably ⅙ or less, most preferably ⅛ or less of the area of the cell group in a state of being seeded on a stimulus-responsive culture substrate. When the contraction ratio of the cell sheet is large, the cell sheet in which cells are dense is obtained, and the characteristics of hepatocytes observed in NASH are remarkably reproduced.

In this description, the “hepatocyte” is a main cell constituting the liver, and is a cell involved in protein synthesis and storage, transformation of carbohydrates, synthesis of cholesterol, bile acid, and phospholipids, as well as detoxification, modification, and excretion of substances, and is also referred to as “liver parenchymal cell”.

The “hepatocyte” used in the presently disclosed subject matter may be a cell isolated from the liver or a part thereof, and may be a primary hepatocyte, a liver precursor cell, a liver stem cell, or an immortalized hepatocyte, or may be a hepatocyte obtained by induction of differentiation from a pluripotent stem cell such as an ES cell, an iPS cell, or a Muse cell, but is preferably a primary hepatocyte.

The “hepatocyte” used in the presently disclosed subject matter is a hepatocyte derived from a mammal (for example, a human, a primate other than humans, a rodent (a mouse, a rat, a hamster, a guinea pig, or the like), a rabbit, a dog, cattle, a horse, a pig, a cat, a goat, a sheep, or the like), more preferably derived from a primate, and is particularly preferably a human hepatocyte. The number of hepatocytes contained in the hepatocyte construct of the presently disclosed subject matter varies depending on the state of the cells, an animal species, a cell type, the number of adherent cells to be co-cultured, or the like, however, for example, the seeding density when the cell sheet to be used for constructing the hepatocyte construct is produced may be from 0.3×10⁴ to 10×10⁶/cm², or may be from 0.5×10⁴ to 8×10⁶/cm², or may be from 0.7×10⁴ to 5×10⁶/cm², or may be from 1.0×10⁴ to 1.0×10⁶/cm², or may be from 5.0×10⁴ to 5.0×10⁵/cm².

In this description, the “adherent cells that are non-hepatocytes” are cells adhered by themselves to an appropriate anchorage for survival, proliferation, and substance production, and are cells also called anchorage-dependent cells, and refer to cells other than hepatocytes. For example, the adherent cells include epithelial cells, interstitial cells, endothelial cells, mucosal cells, fibroblasts, mesenchymal stem cells, pluripotent stem cells, neural stem cells, bone marrow stem cells, germline stem cells, or established cell lines thereof, and the like, but are not limited thereto.

In one embodiment, the adherent cells used in the presently disclosed subject matter are preferably cells that produce a high amount of an extracellular matrix (ECM). The extracellular matrix includes collagen, proteoglycans (for example, chondroitin sulfate proteoglycan, heparan sulfate proteoglycan, keratan sulfate proteoglycan, and dermatan sulfate proteoglycan), hyaluronic acid, fibronectin, laminin, tenascin, entactin, elastin, and fibrillin. And as the adherent cells used in the presently disclosed subject matter, cells that produce any of the above-mentioned extracellular matrices in a higher amount than, for example, a 3T3-J2 strain (for example, primate fibroblasts, mesenchymal stem cells, or the like) can be used. The production amount of an extracellular matrix can be measured with a known method (for example, a quantitative PCR (qPCR), a Western blotting, a flow cytometry (FACS), an ELISA, an immunofluorescent staining, an immunohistochemistry, or the like).

Further, in one embodiment, the adherent cells used in the presently disclosed subject matter are preferably cells having a high contraction ratio when they are released from the anchorage. In this description, the “cells having a high contraction ratio” can be calculated, for example, from the ratio of the area before released to the area after released when the cells are seeded on an arbitrary culture substrate (preferably a stimulus-responsive culture substrate) and cultured to confluency or subconfluency. In one embodiment, as the adherent cells used in the presently disclosed subject matter, for example, cells having a higher contraction ratio than a 3T3-J2 strain (for example, primate fibroblasts, mesenchymal stem cells, or the like) can be used.

In this description, the “fibroblast” is a main cell constituting a connective tissue and is known to produce a collagen fiber (for example, collagen or the like), an elastic fiber (elastin, a microfibril, or the like), a reticular fiber, a substrate (for example, glucosaminoglycan), fibronectin, and the like. In one embodiment, the hepatocyte construct of the presently disclosed subject matter contains fibroblasts of a primate (a human and a primate other than humans), preferably contains human fibroblasts. Further, in one embodiment, the fibroblasts contained in the hepatocyte construct of the presently disclosed subject matter are preferably fibroblasts derived from the dermis.

In this description, the “mesenchymal stem cell” is an undifferentiated cell and refers to a cell having an ability to differentiate into various mesenchymal cells such as an adipocyte, a chondrocyte, an osteocyte, a myoblast, a fibroblast, a stromal cell, and/or a tenocyte, and also having a self-replicating ability. The mesenchymal stem cell is a cell isolated from a tissue such as bone marrow, adipose tissue, umbilical cord blood, dental pulp, synovial membrane, or placenta in a living body, and can be isolated using a known method.

For example, bone marrow-derived mesenchymal stem cells can be isolated as adherent cells by subjecting a bone marrow aspirate collected from the bone marrow to a density gradient centrifugation to separate hematopoietic cells, seeding and culturing the hematopoietic cells on a plastic culture dish at 37° C. in an atmosphere with 5% CO₂.

Adipose tissue-derived mesenchymal stem cells (also referred to as adipose tissue-derived stem cells) (ADSC) can be isolated as adherent cells by, for example, fragmenting collected adipose tissue, digesting the adipose tissue with collagenase type II, adding a culture medium thereto, followed by centrifugation, washing the precipitated cells with a minimal essential medium, filtering the cells through a mesh such as a cell strainer, seeding and culturing the cells on a plastic culture dish at 37° C. in an atmosphere with 5% CO₂. As a method for isolating mesenchymal stem cells derived from another tissue, a known method may be used, and the method is not limited thereto.

The present inventors found that the hepatocyte construct of the presently disclosed subject matter shows characteristics (for example, abundant accumulation of lipid droplets, formation of Mallory-Denk bodies, ballooning, etc.) of hepatocytes in nonalcoholic steatohepatitis (NASH). A hepatocyte construct to be used in vitro showing a plurality of characteristics of NASH has not been developed so far. Therefore, the hepatocyte construct of the presently disclosed subject matter is useful for developing a method or an agent for drastically treating NASH (for example, drug screening). Further, the hepatocyte construct of the presently disclosed subject matter may be used for producing a liver disease model animal, and for example, by transplanting the hepatocyte construct of the presently disclosed subject matter into a non-human mammal (for example, a primate other than humans, a rodent (a mouse, a rat, a hamster, a guinea pig, or the like), a rabbit, a dog, cattle, a horse, a pig, a cat, a goat, a sheep, or the like), a liver disease model animal can be produced.

The number of adherent cells contained in the hepatocyte construct of the presently disclosed subject matter varies depending on the state of the cells, an animal species, a cell type, the number of hepatocytes to be co-cultured, or the like, however, for example, the seeding density when a co-culture cell sheet thereof with the hepatocytes is produced may be from 0.3×10⁴ to 10×10⁶/cm², or may be from 0.5×10⁴ to 8×10⁶/cm², or may be from 0.7×10⁴ to 5×10⁶/cm², or may be from 1.0×10⁴ to 1.0×10⁶/cm², or may be from 5.0×10⁴ to 5.0×10⁵/cm².

In one embodiment, the ratio of the hepatocytes to the adherent cells used when the hepatocyte construct of the presently disclosed subject matter is produced may be, for example, in a range of 10:1 to 1:10, preferably in a range of 5:1 to 1:10, more preferably in a range of 1:1 to 1:10.

In this description, the “ballooned hepatocyte” is one of the appearance characteristics of a hepatocyte seen when the liver of a patient who suffers from nonalcoholic steatohepatitis (NASH) is subjected to biopsy, and refers to a hepatocyte that is expanded like a balloon as compared with a normal hepatocyte. The hepatocyte construct of the presently disclosed subject matter contains ballooned hepatocytes observed in NASH.

In this description, the “Mallory-Denk body” refers to an inclusion body in an eosinophilic cytoplasm in which an ubiquitinated protein, keratin 8 and 18, which is an intermediate filament, p62, or the like confirmed in nonalcoholic steatohepatitis (NASH) or a tissue of a hepatocellular cancer patient is accumulated as a structure in an indeterminate form. In one embodiment, the hepatocytes contained in the hepatocyte construct of the presently disclosed subject matter contains Mallory-Denk bodies observed in NASH. The Mallory-Denk bodies can be visualized by a known method, but can be visualized by, for example, staining using an antibody against keratin 8 and 18.

In one embodiment, the hepatocyte construct of the presently disclosed subject matter is characterized in that the expression level of sonic hedgehog protein (hereinafter referred to as “SHH”) per cell is high. SHH is a factor well known to a person skilled in the art as a hedgehog monologue involved in a hedgehog signal transduction pathway, and for example, although not limited to, a nucleic acid sequence (mRNA) and an amino acid sequence of a human SHH gene are provided under the accession numbers in GenBank database and GenPept database of NM_000193 and NM_001310462, and NP_000184 and NP_001297391. That is, the sequence of “SHH” can be obtained by a person skilled in the art from, for example, GenBank database and GenPept database according to the biological species of the cells to be used, and the sequences thereof and the sequences of variants thereof are also included in the scope of SHH of the disclosed subject matter of this application. It is considered that SHH acts as a paracrine pro-fibrogenic factor for hepatic stellate cells in the liver, thereby inducing activation of myofibroblasts and causing fibril formation. In the hepatocyte construct of the presently disclosed subject matter, the expression level of SHH is significantly higher than that in normal hepatocytes, and this reproduces the characteristic of hepatocytes derived from NASH. The expression level of SHH can be measured by a known method, and, for example, the measurement can be performed using a well-known technique such as a quantitative PCR (qPCR), a Western blotting, a flow cytometry (FACS), an ELISA, an immunofluorescent staining method, or an immunohistochemistry.

In one embodiment, the hepatocyte construct of the presently disclosed subject matter expresses α-SMA (also referred to as ACTA2/smooth muscle actin) that is an activation marker for myofibroblasts. For example, although not limited to, a nucleic acid sequence (mRNA) and an amino acid sequence of a human α-SMA gene are provided under the accession numbers in GenBank database and GenPept database of NM_001141945, NM_001613, and NM_001320855, and NP_001135417, NP_001307784, and NP_001604. That is, the sequence of “α-SMA” can be obtained by a person skilled in the art from, for example, GenBank database and GenPept database according to the biological species of the cells to be used, and the sequences thereof and the sequences of variants thereof are also included in the scope of α-SMA of the disclosed subject matter of this application. In the hepatocyte construct of the presently disclosed subject matter, α-SMA becomes positive particularly in fibroblasts around ballooned hepatocytes, and this also reproduces the characteristic of a liver tissue derived from NASH. The expression of α-SMA can be measured by a known method, and, for example, the measurement can be performed using a well-known technique such as a quantitative PCR (qPCR), a Western blotting, a flow cytometry (FACS), an ELISA, an immunofluorescent staining, or an immunohistochemistry.

In one embodiment, the hepatocyte construct of the presently disclosed subject matter may contain cells other than the above-mentioned cells, and for example, may contain hepatic stellate cells, pericytes, endothelial cells, or smooth muscle cells, or a combination thereof.

### <<Method for Producing Hepatocyte Construct>>

The presently disclosed subject matter provides a method for producing a hepatocyte construct, and includes:

(i) forming an aggregate by aggregating a cell group containing hepatocytes and adherent cells that are non-hepatocytes; and

(ii) culturing the aggregate. By culturing the aggregate obtained in the step (i) in the step (ii), a hepatocyte construct comprising the aggregate containing hepatocytes and adherent cells that are non-hepatocytes, wherein the hepatocytes include ballooned hepatocytes, can be produced.

In one embodiment, the step (i) may include a step of seeding the hepatocytes and the adherent cells that are non-hepatocytes on a substrate to which cells are not adhered (for example, a Petri dish or the like), and culturing them while allowing a centrifugal force to act toward the bottom face of the substrate (see, for example, Japanese Patent No. 5407343).

In one embodiment, the step (i) may include, for example, the following steps:

(i-1) seeding the hepatocytes on a first culture substrate and performing culture;

(i-2) seeding the adherent cells on the first culture substrate on which the hepatocytes are seeded and performing culture; and

(i-3) releasing the cell group containing the hepatocytes and the adherent cells from the first culture substrate, thereby forming the aggregate.

In this case, the first culture substrate may be, for example, a “stimulus-responsive culture substrate”.

In this description, the “stimulus-responsive culture substrate” refers to a cell culture substrate coated with a polymer that changes the molecular structure by a stimulus such as temperature, pH, light, or electricity. Arbitrary cells are seeded on the stimulus-responsive culture substrate and cultured until the cells become confluent or sub confluent, and thereafter, the stimulus-responsive culture substrate is changed by changing the condition of the stimulus such as temperature, pH, light, or electricity, whereby the cell group is released in a sheet form from the stimulus-responsive culture substrate while maintaining the state where the cells are adhered to one another, and the aggregate can be obtained. In order to more quickly released the aggregate, a method in which the stimulus-responsive culture substrate is lightly tapped or shaken, a method in which the culture medium is stirred using a pipette, a method in which forceps are used, and the like may be used singly or in combination.

In this description, a “stimulus-responsive polymer” which is coated on the stimulus-responsive culture substrate includes but not limited to poly(N-isopropylacrylamide), a poly(N-isopropylacrylamide-acrylic acid) copolymer, a poly(N-isopropylacrylamide-methyl methacrylate) copolymer, a poly(N-isopropylacrylamide-sodium acrylate) copolymer, a poly(N-isopropylacrylamide-vinylferrocene) copolymer, poly(vinyl methyl ether) (PVME) irradiated with a γ ray, poly(oxyethylene), a resin in which a biological substance such as a nucleic acid is integrated into a polymer, and a gel produced by crosslinking the above-mentioned polymer with a crosslinking agent.

In one embodiment, the stimulus-responsive culture substrate that can be used as the first culture substrate may be, for example, a temperature-responsive culture substrate. In this description, the “temperature-responsive culture substrate” refers to a cell culture substrate coated with a temperature-responsive polymer. In this description, the “temperature-responsive polymer” is one of the stimulus-responsive polymers and refers to a polymer that changes its form and/or property in response to temperature. The temperature-responsive polymer includes but not limited to poly(N-isopropylacrylamide), a poly(N-isopropylacrylamide-acrylic acid) copolymer, a poly(N-isopropylacrylamide-methyl methacrylate) copolymer, a poly(N-isopropylacrylamide-sodium acrylate) copolymer, a poly(N-isopropylacrylamide-vinylferrocene) copolymer, poly(vinyl methyl ether) irradiated with a γ ray, and a gel produced by crosslinking the above-mentioned polymer with a crosslinking agent. Preferably, for example, poly(N-isopropylacrylamide), a poly(N-isopropylacrylamide-methyl methacrylate) copolymer, a poly(N-isopropylacrylamide-sodium acrylate) copolymer, and a material formed by crosslinking the above-mentioned polymer with a crosslinking agent are exemplified, but it is not limited thereto.

In one embodiment, as the temperature-responsive polymer which is coated on the first culture substrate, for example, a polymer having an upper critical solution temperature (UCST) or a lower critical solution temperature (LCST) in water of 0 to 80° C. is exemplified, but it is not limited thereto. In this description, the critical solution temperature refers to a threshold temperature at which the form and/or property of the polymer is changed. In one embodiment of the presently disclosed subject matter, the first culture substrate may be a temperature-responsive culture substrate in which poly(N-isopropylacrylamide) (PIPAAm) is coated on at least a part of the culture face thereof.

The poly(N-isopropylacrylamide) (PIPAAm) is a polymer having a lower critical solution temperature (LCST) of 32° C., and if it is in a free state, dehydration is caused at a temperature of 32° C. or higher in water, and the aggregation and white turbidity of the polymer occurs. On the other hand, PIPAAm is hydrated at a temperature lower than 32° C., and becomes in a state of being dissolved in water. The temperature-responsive culture substrate that can be used in one embodiment of the presently disclosed subject matter is a substrate in which PIPAAm is coated on a culture substrate such as a dish and is fixed thereto. Therefore, when the temperature is 32° C. or higher, PIPAAm on the culture surface is dehydrated, and the surface of the culture substrate becomes hydrophobic. On the other hand, when the temperature is lower than 32° C., PIPAAm on the surface of the culture substrate is hydrated and the surface of the culture substrate becomes hydrophilic. To the hydrophobic surface of the culture substrate, the cells are adhered and can be proliferated thereon, and further, the hydrophilic surface of the culture substrate is a surface to which the cells are not easily adhered. Therefore, when the temperature-responsive culture substrate is cooled to a temperature lower than 32° C., the cells are noninvasively released from the surface of the culture substrate.

In one embodiment, the step (ii) is adhering the aggregate onto a second culture substrate and culturing it. By adhering the aggregate onto the second culture substrate and performing culture, transformation of hepatocytes into ballooned hepatocytes is promoted. In this description, the “second culture substrate” is not particularly limited as long as it is a substrate capable of culturing it while having the adherent cells adhered thereto, however, for example, a dish, a multiplate, a flask, a flat film-like substrate, or the like can be used.

In one embodiment, the culture substrate (the first culture substrate and/or the second culture substrate) used in the presently disclosed subject matter may be coated with a cell-adhesive hydrogel. In this description, the “cell-adhesive hydrogel” refers to a material selected from the group consisting of an extracellular matrix component or a chitosan gel, a collagen gel, gelatin, a peptide gel, a laminin gel, and a fibrin gel, and a mixture thereof. Among these, a chitosan gel, a collagen gel, gelatin, a peptide gel, and a laminin gel are gelled by, for example, changing the temperature, pH, and/or salt concentration. A fibrin gel is gelled when fibrinogen, which is a monomer, acts with thrombin, which is an enzyme. The cell-adhesive hydrogel used in the presently disclosed subject matter is preferably a collagen gel. In this description, collagen contained in the collagen gel is, for example, type I, type II, type III, type IV, type V, type VI, type VII, type VIII, type IX, type X, type XI, type XV, type XVII, or type XVIII collagen or atelocollagen, or a combination thereof. The collagen contained in the collagen gel may be derived from a mammal (for example, a primate (including a human and a primate other than humans), a rodent (a mouse, a rat, a hamster, a guinea pig, or the like), a rabbit, a dog, cattle, a horse, a pig, a cat, a goat, a sheep, or the like), or may be collagen obtained through genetic engineering based on a collagen gene of a mammal.

In one embodiment, the ratio of the “hepatocytes” to the “adherent cells that are non-hepatocytes” used in the step (i) varies depending on the state of the cells, an animal species, a cell type, or the like, but may be, for example, from 10:1 to 1:10, and is preferably in a range of 5:1 to 1:10, more preferably in a range of 1:1 to 1:10. In one embodiment, the number of “hepatocytes” to be seeded varies depending on the state of the cells, an animal species, a cell type, the number of “adherent cells that are non-hepatocytes” to be co-cultured, or the like, but, for example, may be from 0.3×10⁴ to 10×10⁶/cm², or may be from 0.5×10⁴ to 8×10⁶/cm², or may be from 0.7×10⁴ to 5×10⁶/cm², or may be from 1.0×10⁴ to 1.0×10⁶/cm², or may be from 5.0×10⁴ to 5.0×10⁵/cm². In one embodiment, the number of “adherent cells that are non-hepatocytes” to be seeded varies depending on the state of the cells, an animal species, a cell type, the number of hepatocytes to be co-cultured, or the like, but, for example, may be from 0.3×10⁴ to 10×10⁶/cm², or may be from 0.5×10⁴ to 8×10⁶/cm², or may be from 0.7×10⁴ to 5×10⁶/cm², or may be from 1.0×10⁴ to 1.0×10⁶/cm², or may be from 5.0×10⁴ to 5.0×10⁵/cm².

In one embodiment, it is preferred that the “adherent cells that are non-hepatocytes” to be seeded are seeded at a density such that the aggregate after being released is contracted to an area that is ¼ or less, more preferably ⅕ or less, further more preferably ⅙ or less, most preferably ⅛ or less of the area of the cell group containing the hepatocytes and the “adherent cells that are non-hepatocytes” before being released from the first cell culture substrate. When the contraction ratio of the aggregate is large, the aggregate in which the cells are denser is obtained, and the characteristics of the hepatocytes observed in NASH can be remarkably reproduced.

As the culture medium used in the method of the presently disclosed subject matter, a known culture medium capable of culturing hepatocytes may be used, and for example, Dulbecco's modified Eagle's medium (DMEM), minimal essential medium (MEM), knockout-DMEM (KO-DMEM), Glasgow minimal essential medium (G-MEM), Eagle's minimal essential medium (BME), DMEM/Ham's F12, Advanced DMEM/Ham's F12, Iscove's modified Dulbecco's medium and minimal essential medium (MEM), Ham's F-10, Ham's F-12, 199 medium, and RPMI 1640 medium, etc. are exemplified, but it is not limited thereto.

A preferred culture medium is a high-glucose culture medium supplemented with insulin and transferrin. Further, to the culture medium, serum (for example, fetal bovine serum) may be added, and it may be serum-free culture. In one embodiment, a high-glucose containing culture medium used in the presently disclosed subject matter contains glucose at 1 mM to 100 mM, preferably at 5 mM to 80 mM, more preferably at 10 mM to 50 mM, for example, at about 25 mM. Further, the culture medium used in the presently disclosed subject matter contains insulin at 0.1 μM to 10 μM, preferably at 0.5 μNI to 5 μM, and for example, at about 1 μM. Containing high glucose and/or insulin in the culture medium allows the characteristics of the hepatocytes observed in NASH to be more efficiently reproduced.

In one embodiment, the culture time in the step (i-1) may be time enough for the hepatocytes to sufficiently adhere to the first culture substrate, and is not particularly limited, but is for example, from 3 hours to 72 hours, preferably from 6 hours to 48 hours, more preferably from 12 hours to 36 hours, and for example, about 24 hours.

In one embodiment, the culture time in the step (i-2) may be time enough for the “adherent cells that are non-hepatocytes” to sufficiently adhere to the first culture substrate and become confluent or subconfluent so that a cell sheet can be formed, and is not particularly limited, but is for example, from 24 hours to 120 hours, preferably from 36 hours to 96 hours, more preferably from 24 hours to 84 hours, and for example, about 72 hours.

In one embodiment, the culture time in the step (ii) may be time enough for the hepatocytes contained in the aggregate to be cultured to such an extent that the characteristics of the hepatocytes observed in NASH, for example, ballooning, accumulation of Mallory-Denk bodies, abundant accumulation of lipid droplets, fibril formation around the hepatocytes, appearance of α-SMA-positive cells, etc. are observed, and is not particularly limited, however, the culture may be performed, for example, for 3 days, 4 days, 5 days, 6 days, 7 days, 8 days, 9 days, 10 days, 11 days, 12 days, 13 days, 14 days, 15 days, or 15 days or more.

### <<Method for Evaluating Factor for Treating or Preventing Liver Disease>>

The hepatocyte construct of the presently disclosed subject matter or a hepatocyte construct obtained by the method of the presently disclosed subject matter can be used in a method for evaluating a factor for treating or preventing a liver disease. For example, in one embodiment, the method of the presently disclosed subject matter comprises

(1) applying a candidate factor to the hepatocyte construct and culturing it, and

(2) evaluating a therapeutic or preventive effect of the candidate factor using a characteristic (for example, abundant accumulation of lipid droplets, formation of Mallory-Denk bodies, ballooning, or the like) of the hepatocytes in nonalcoholic steatohepatitis (NASH) as an index in the hepatocyte construct.

In one embodiment, the candidate factor applied in the step (1) may be applied to the hepatocyte construct (for example, added to the culture medium), or may be applied in an arbitrary step of the above-mentioned method for producing a hepatocyte construct (for example, added to the culture medium).

In one embodiment, the characteristic (for example, abundant accumulation of lipid droplets, formation of Mallory-Denk bodies, ballooning, or the like) of the hepatocytes in nonalcoholic steatohepatitis (NASH) in the step (2) can be evaluated using an arbitrary microscope after staining the hepatocyte construct using a known staining method (for example, although not limited to, hematoxylin-eosin (H&E) staining, nile red staining, immunohistochemical staining or immunofluorescent staining of keratin, or the like).

In one embodiment, the candidate factor includes a low molecular weight compound, a peptide, a nucleic acid, a protein, a cell extract or a tissue extract of a mammal (for example, a mouse, a rat, a pig, cattle, a sheep, a monkey, a human, or the like), or a cell culture supernatant, a compound or an extract derived from a plant (for example, a herbal medicine extract or a compound derived from a herbal medicine), a compound or an extract derived from a microorganism, and a culture product.

### EXAMPLES

Hereinafter, the presently disclosed subject matter will be described in more detail based on Examples, however, these are not intended to limit the presently disclosed subject matter.

### Example 1

### 1. Materials & Methods

### 1-1. Cells

Normal human dermal fibroblasts (NHDF; Lonza, Basel, Switzerland) were maintained in high-glucose Dulbecco's Modified Eagle's Medium (DMEM, Coming, N.Y., US) supplemented with 10% fetal bovine serum (FBS; ThermoFisher Scientific) and 1% penicillin-streptomycin (Coming) and cultured at 37° C. in a humidified atmosphere with 5% CO₂. NHDFs were passaged at preconfluency by trypsinization. NHDFs from passages 3 to 7 were used for the co-culture experiments.

Mouse 3T3-J2 fibroblasts (Kerafast, Boston, Mass., US) were maintained in high-glucose DMEM supplemented with 10% bovine calf serum (GE Healthcare, Little Chalfont, UK) and 1% penicillin-streptomycin (Coming) and cultured at 37° C. in a humidified atmosphere with 5% CO₂. Cells were passaged at preconfluency by trypsinization. Cells from passages 3 to 5 were used for the co-culture experiments.

Primary human hepatocytes (PHH) (Lot: Hu8200A and Hu1652) were purchased from ThermoFisher Scientific. On the basis of the instructions provided by the supplier, PHHs were thawed and viability was assessed using the trypan blue. For the two lots, viability was more than 90%.

### 1-2. Primary Human Hepatocytes/Fibroblasts Co-Cultures

Temperature responsive cell culture 24 well plates (UpCell™; CellSeed, Tokyo, Japan) were coated with 100 μg/ml rat tail collagen I solution (Corning) overnight. Then the UpCell 24 well plates were washed with phosphate-buffered saline (PBS) for two times.

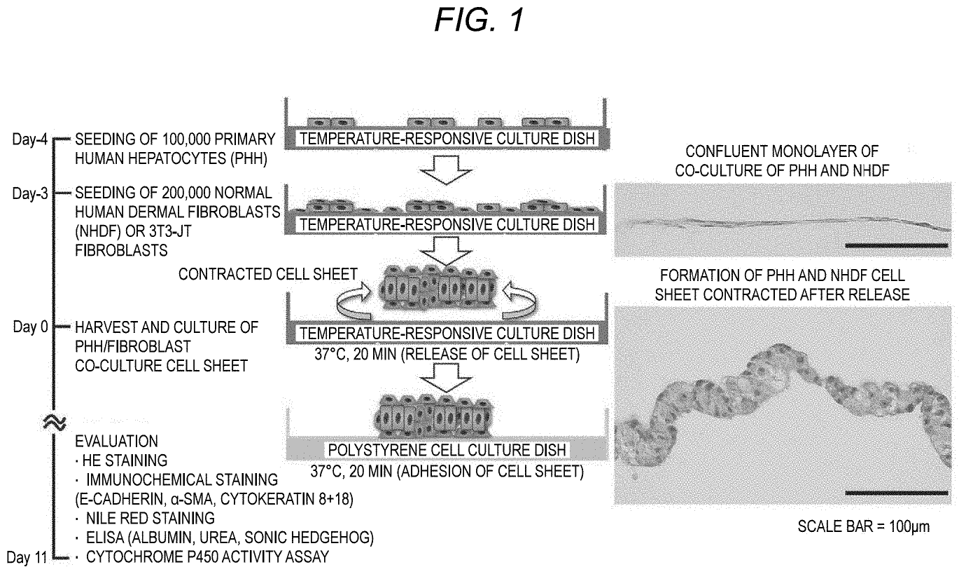

As shown in FIG. 1, PHHs were seeded into collagen coated UpCell 24 well plates at 1×10⁵ cells/well (day −4). Hepatocytes were cultured with high-glucose DMEM supplemented with 0.1 μM dexamethasone (Sigma Aldrich, St. Louis, Mo.), 1% ITS premix (insulin/human transferrin/selenous acid and linoleic acid; Corning), 0.2 μM glucagon (Sigma Aldrich), 10% FBS, 1% Penicillin-streptomycin at 37° C. in a humidified atmosphere with 5% CO2.

After culturing PHHs overnight (day −3), two types of fibroblasts (3T3-J2 and NHDF) were seeded at 2×10⁵ cells/well in hepatocyte culture medium to create co-cultures. Culture media was replaced daily until day 0.

### 1-3. Production of Hepatocyte Construct

On day 0, confluent PHH/fibroblast co-cultures on UpCell™ plates were incubated at 20° C. for 30 min to be released as free-floating cell sheets. Release of a monolayer of confluent cells resulted in rapid contraction which transformed a thin monolayer of cells into a thick contracted cell sheet with multilayered structure (FIG. 1).

These free floating cell sheets were transferred into collagen I-coated 35 mm polystyrene cell culture dishes (IWAKI, Tokyo, Japan) by 10 ml pipettes. Then cell culture media was drained and the cell sheets were incubated at 37° C. without media to attach on collagen coated surfaces. Around 10 min later, 1 ml hepatocyte culture media (5% FBS) was added into cell culture dishes. The media was replaced daily until day 11. Cell sheets were observed and imaged using a phase-contrast microscope (Nikon, Tokyo, Japan)

### 1-4. Immunohistochemistry (IHC) Staining

At pre-determined time points, cell sheet samples were washed by pre-warmed PBS twice and fixed in 4% paraformaldehyde at room temperature for 1 h. Fixed cell sheets were embedded in paraffin, sliced into 4 μm sections and deparaffinized for standard histological staining with hematoxylin and eosin (HE). For immunostainings of cytokeratin 8+18 (CK8/18), sections were treated with Dako proteinase K (Agilent, Santa Clara, Calif., US) for antigen retrieval, incubated in Dako REAL peroxidase-blocking solution (Agilent) to quench endogenous peroxidase activity, and then blocked in Blocking One Histo (Nacalai Tesque, Kyoto, Japan). After that, the sections were incubated with mouse anti-human CK8/18 at 25-fold dilution (Abeam, Cambridge, UK) followed by incubation with horseradish peroxide-conjugated donkey anti-mouse IgG H&L (Abeam). The sections were dyed with Dako Liquid DAB+Substrate Chromogen System (Agilent) and the nuclei were stained with hematoxylin. Finally, the sections were mounted, dried and imaged with a light microscope (Nikon).

### 1-5. Immunofluorescence Staining

In brief, paraffin-embedded sections were incubated with primary antibodies as follows: E-Cadherin (Abeam), α-smooth muscle actin (α-SMA) (Abeam), and CK8/18 (Abeam). Alexa Fluor 488 goat anti-rabbit and Alexa Fluor 594 goat anti-mouse were used as secondary antibodies for staining. The cell nuclei were stained with 4′,6-diamidino-2-phenylindole (DAPI). Finally, the sections were mounted, dried and imaged with a confocal laser scanning microscope (CLSM; Olympus, Tokyo, Japan) or a fluorescence microscope (Nikon).

On the E-Cadherin/DAPI staining images, PHHs with positive DAPI staining were chosen for quantification of cross-section area per PHH by Image J software. Measurement was performed in three independent experiments. In each experiment, more than 25 cells were measured.

### 1-6. Nile Red Staining

In order to stain cytoplasmic lipid droplets, fixed cell sheet samples on day 11 were washed with PBS for two times and incubated with 1 μg/ml Nile red (Sigma Aldrich) for 30 min. Then, images of immunofluorescently labeled lipids were acquired with a CLSM (Olympus).

### 1-7. Enzyme-Linked Immunosorbent Assay (ELISA) Analysis

At pre-determined time points, cell culture supernatants over a 24 h period were collected and stored at −30° C. until assayed. Secretion of albumin from hepatocytes was detected by human albumin ELISA quantitation kit (Bethyl Laboratories, Montgomery, Tex., US). Urea synthesis by cultured hepatocytes was determined using a colorimetric assay kit (BioChain, Newark, Calif., US). The amount of SHH ligands produced by hepatocytes was determined by human SHH ELISA kit (Abeam). Measurements were performed in three independent experiments. In each experiment, replicate samples were more than three.

### 1-8. Cytochrome P450 (CYP) Enzyme Activities

Briefly, cell sheet samples were washed with PBS and then replaced with fresh media containing 100 μM phenacetin (PHE; Sigma Aldrich) and 50 μM midazolam (MDZ; Sigma Aldrich), which were substrate of CYP1A2 and CYP3A4, respectively. PHHs in cell sheets adsorbed and metabolized two CYP substrates and released their metabolites into culture media. After 10 min incubation, 20 μl cell culture supernatants were collected. They contained metabolites of PHE and MDZ, which were acetaminophen and hydroxymidazolam, respectively. The two metabolites were measured by API 5000 LC/MS/MS System (AB applied biosystems, Foster City, Calif., US). Biotransformation rate of CYP substrates into specific metabolites was used to indicate CYP enzyme activities. Measurement of CYP activities was performed in three independent experiments using two lots of PHH (Lot8200_A×2+Lot1652×1). In each experiment, replicate samples were more than three.

### 1-9. Statistical Analysis

At each experiment, values from at least three replicate samples were obtained. All the experiments were repeated for three or four times with two lots of PHHs. Data were expressed as mean±standard deviation (SD). Significant differences between two groups were tested by Student's t-test. Significant differences of cross-section area per PHH at three pre-determined time points were tested using analysis of variance (ANOVA) followed by a Dunnett's post hoc test, using IBM SPSS Statistics 25 software. Values of p<0.05 were considered to be statistically significant.

### 2. Results

### 2-1. Morphological Findings of PHH/Fibroblast Co-Culture Cell Sheets

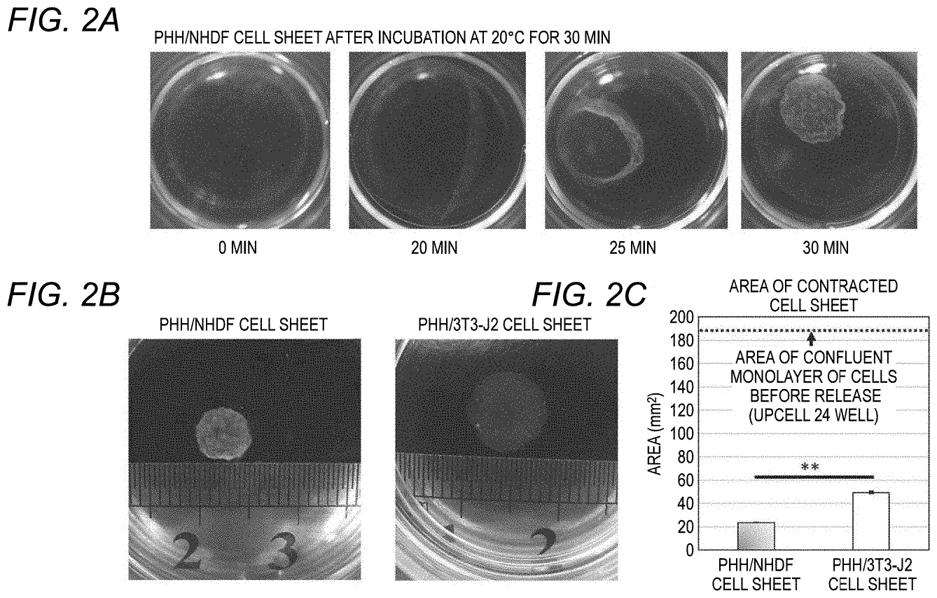

After harvesting a two-dimensional (2D) co-culture of PHHs and fibroblasts as a free floating cell sheet, since restriction force from underlying dish disappeared, free cell sheets underwent rapid and symmetric contraction (reduction in area but increase in thickness) (FIG. 2A). This contractile force was driven by cytoskeletal change of cells in the intact cell sheets during the releasing process. Elongated cells became compacted and thus contractive force was generated to form a 3D multilayered assembly. The extent of shrinkage depended on the contractive abilities of cells. As shown in FIG. 2C, the area of PHH/NHDF cell sheets is around 24 mm², which is only ⅛ of its original area (190 mm²) before release. Whereas, the area of PHH/3T3-J2 cell sheet is 49 mm² which is two times bigger than that PHH/NHDF cell sheets (FIGS. 2B and 2C). We considered this was probably attributed to stronger contractive ability of NHDFs.

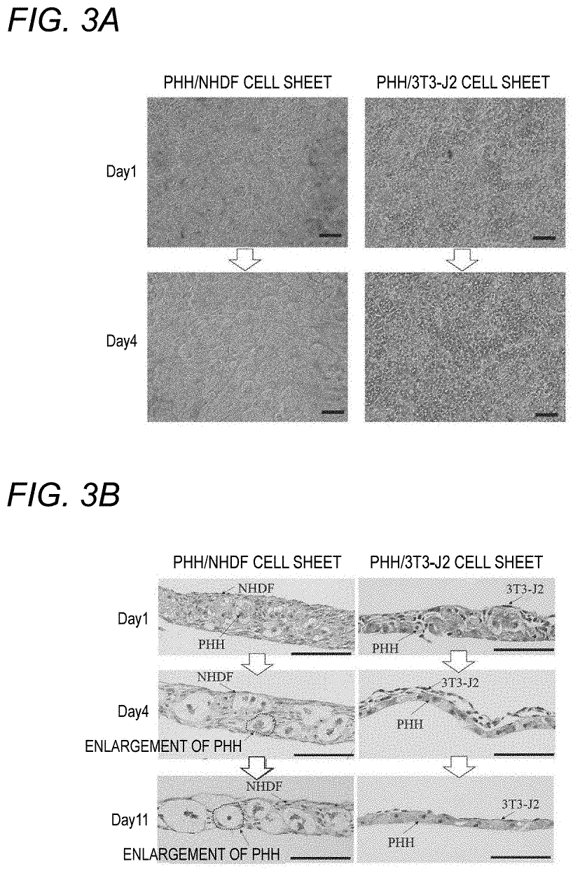

In accordance with the macroscopic findings, phase-contrast microscopy also revealed a very compacted architecture in the PHH/NHDF cell sheet, in which cells were bunched up together forming many small clumps (FIG. 3A, left top). Surprisingly, the clumps seemed to be swollen after 4 days culture (FIG. 3A, left bottom). In contrast, cell clumps were not observed in the PHH/3T3-J2 cell sheet. Actually, PHHs in the PHH/3T3-J2 cell sheet showed a very healthy morphology, a cuboidal shape with distinct demarcated cell borders after 4 days culture (FIG. 3A, right bottom).

HE staining also demonstrated a cell dense structure in PHH/NHDF co-culture cell sheets, where PHHs were compacted by densely packed NHDFs (FIG. 3B, left). Corresponding to the phase-contrast microscopy observations, PHHs demonstrated pronounced cellular enlargement with pale staining in the cytoplasm on day 4 and day 11, whereas PHHs in the PHH/3T3-J2 cell sheet became flat with positive eosin staining in the cytoplasm on day 4 and day 11 (FIG. 3B, right).

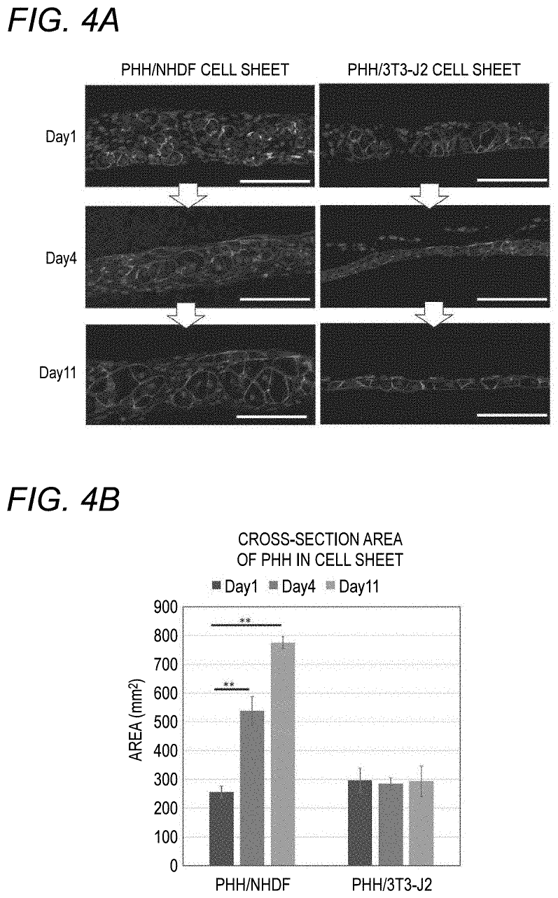

Immunofluorescence staining of E-Cadherin showed clear cell-cell borders of PHHs (FIG. 4A) and thus cross-section area of each PHH were quantified. A 2-fold increase in the cross-section area of each PHH was observed in the PHH/NHDF cell sheet on day 4 relative to day 1 and even a 3-fold increase was observed on day 11 (FIG. 4B). However, the size of PHHs in the healthy PHH/3T3-J2 cell sheet did not changed significantly during 11 days culture (FIG. 4B).

2-2. Enlarged Hepatocytes in PHH/NHDF Co-Culture Cell Sheet were Ballooned Hepatocytes

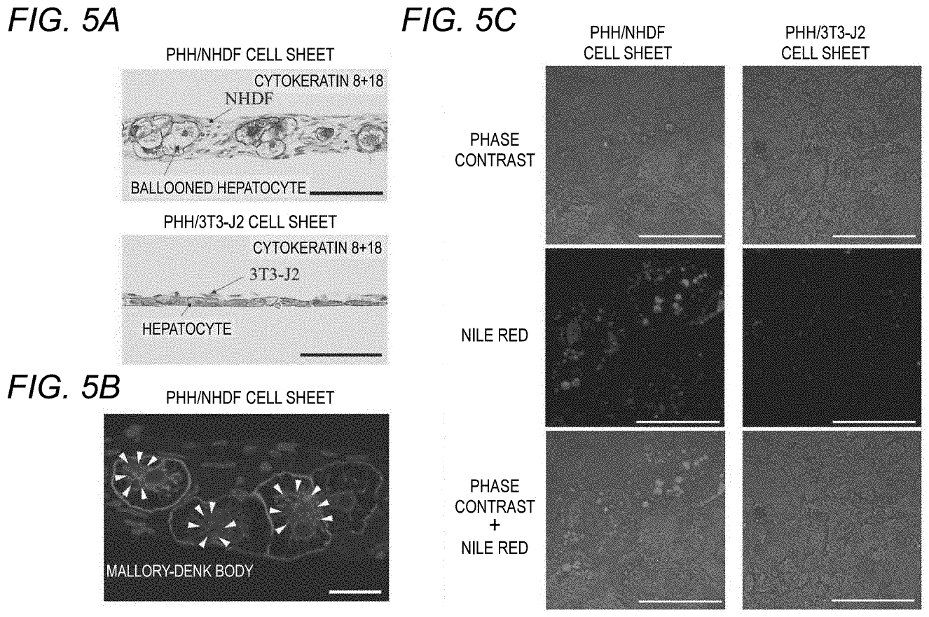

Since the cytoplasm of enlarged hepatocytes were negative for eosin staining, we thought they might be ballooned hepatocytes. To support this hypothesis, IHC staining of CK 8/18 staining were performed. As expected, enlarged hepatocytes in PHH/NHDF cell sheets showed a disturbance, decrease or even loss of the cytoplasmic keratin staining, while substantial decrease or loss of CK 8/18 immunostaining was not observed in normal-sized hepatocytes of PHH/3T3-J2 sheets (FIG. 5A).

Moreover, immunofluorescence staining of CK 8/18 clearly revealed that irregularly shaped CK 8/18 positive cytoplasmic inclusions located in proximity to the nucleus in the enlarged hepatocytes (FIG. 5B), which supported the presence of MDBs. Generally, MDBs is considered to be a histological feature of NASH and its formation is consequence of cytoskeletal damage and associated ballooning degeneration.

Besides MDBs, abundant accumulation of lipid droplets was also detected in the PHH/NHDF cell sheet after staining with Nile red on day 11, while a limited amount of lipid was visible in the PHH/3T3-J2 cell sheet (FIG. 5C).

On the basis of above results, it was suggested that the enlarged PHHs in PHH/NHDF cell sheets were ballooned hepatocytes.

### 2-3. Increased Secretion of SHH and Myofibroblast Activation in PHH/NHDF Co-Culture Cell Sheets

It has been demonstrated that SHH ligands production correlates to hepatocellular ballooning and fibrosis (Guy C. D., et al., Hepatology. 2012 June; 55(6): 1711-1721; Ranagwala F., et al., J. Pathol. 2011, July; 224(3): 401-410). The SHH ligands produced by ballooned hepatocytes act as paracrine pro-fibrogenic factors for hepatic stellate cells and fibroblasts thereby induce their myofibroblast activation and cause fibrogenesis.

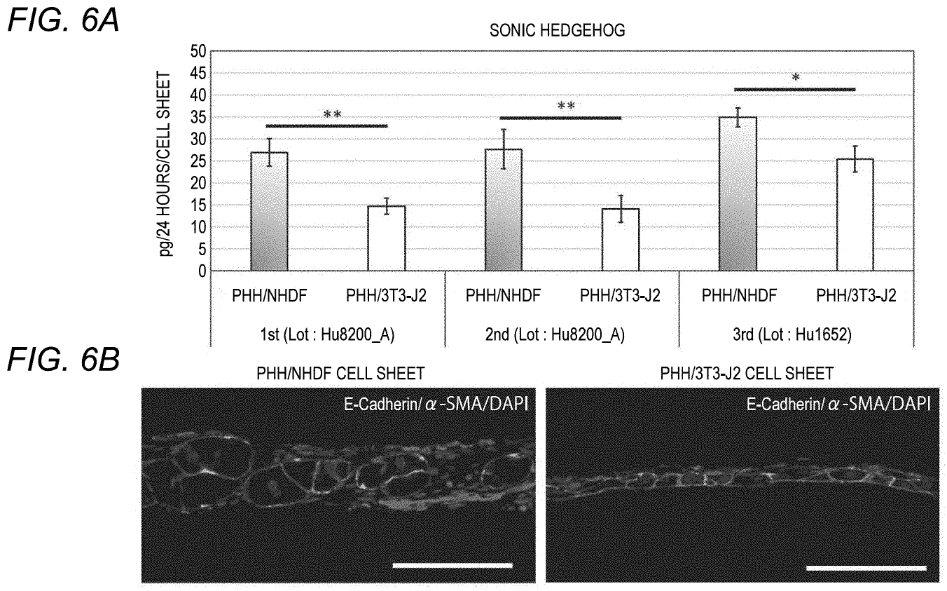

In our in vitro model, we found significant increases of SHH production (1.4-fold in Hu1652 and 2.0-fold in Hu8200_A) in the PHH/NHDF cell sheet compared with the PHH/3T3-J2 cell sheet on day 4 (FIG. 6A). These results corresponded with the myofibroblast activation in the PHH/NHDF cell sheet, where u-SMA (myofibroblast marker) positive NHDFs were observed to be around enlarged hepatocytes (FIG. 6B). On the contrary, the PHH/3T3-J2 cell sheet had a total absence of α-SMA. These findings suggested that our model not only reflected typical hallmarks of ballooned hepatocytes, but also showed the myofibroblast activation, which was also found in human NASH.

### 2-4. Hepatocyte Functions in PHH/Fibroblast Co-Culture Cell Sheets

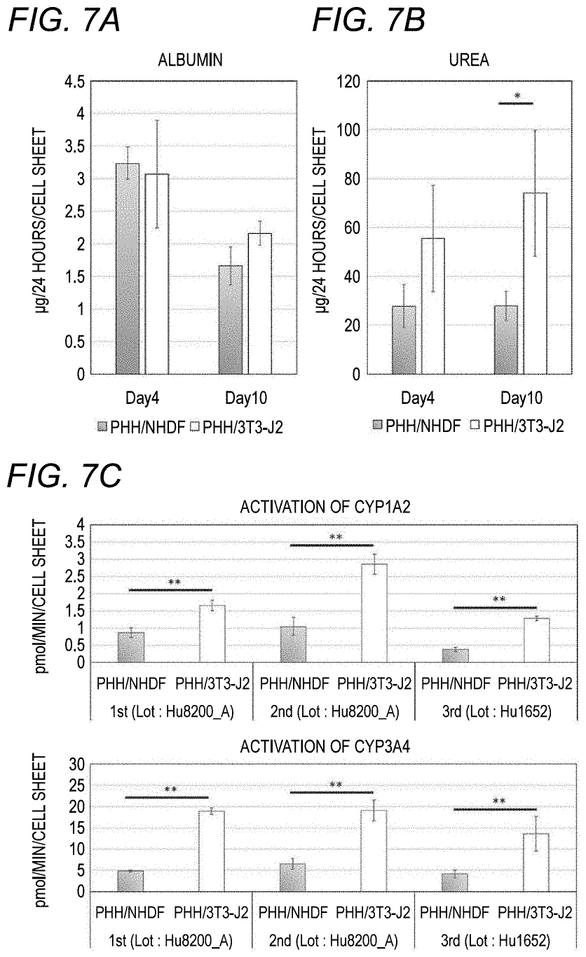

As shown in FIG. 7A, there are no significant difference between PHH/NHDF cell sheets and PHH/3T3-J2 cell sheets in the albumin production no matter on day 4 or day 10. This result suggested that ballooned hepatocytes in PHH/NHDF cell sheets were not only abnormal cells but still capable of maintaining synthesis functions.

As for urea production, there were no significant differences between PHH/NHDF cell sheets and PHH/3T3-J2 cell sheet on day 4, but PHH/3T3-J2 cell sheets showed a 2.6-fold increase compared with PHH/NHDF cell sheets on day 10 (FIG. 7B). Decreased urea production has been reported to be at low oxygen partial pressures (Bhatia S. N., et al., J. Cell Eng. 1996,1,125), so we considered the relatively lower urea production in PHH/NHDF cell sheets on day 10 might be attributed to hypoxia in the thicker PHH/NHDF cell sheets compared with PHH/3T3-J2 cell sheet (FIG. 3B).

However, CYP enzyme activities including CYP1A2 and CYP3A4 were significant lower in PHH/NHDF cell sheets than those in PHH/3T3-J2 cell sheets (FIG. 7C). These findings aligned with a previous study of clinical samples from NAFLD patients, which has shown that CYP1A2 and CYP3A4 activities decreased with disease progression (Fisher C. D., et al., Drug Metab. Dispos. 2009, 37(10), 2087-94).

### 2-5. Co-Culture Ratio of PHH and NHDF

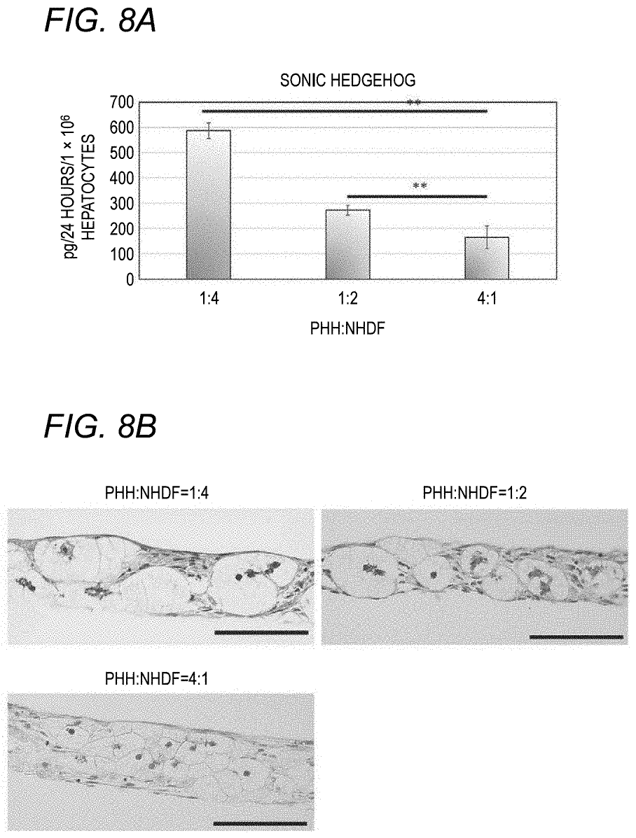

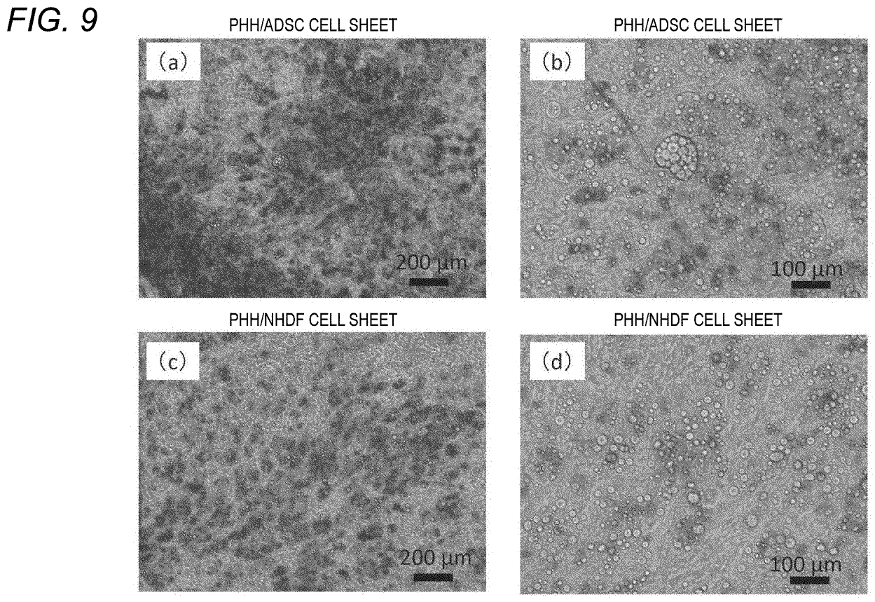

It was examined whether the co-culture ratio of PHHs and NHDFs affects the degree of hepatocellular damages and degeneration (FIGS. 8A and 8B). As a result, it was revealed that SHH production increased 3.5-fold in 1:4 (PHH:NHDF) co-culture and 1.6-fold in 1:2 (PHH:NHDF) co-culture as compared with that in 4:1 (PHH:NHDF) co-culture (FIG. 8A). It was revealed that the size of the hepatocytes in 4:1 co-culture was smaller than that in 1:2 and 1:4 (PHH:NHDF) co-culture (FIG. 8B).

### Example 2

### 1. Experimental Materials & Methods

### 1-1. Cells

Adipose-derived mesenchymal stem cells (ADSC; LONZA) were maintained in high-glucose Dulbecco's Modified Eagle's Medium (DMEM, Corning, N.Y., US) supplemented with 10% fetal bovine serum (FBS; ThermoFisher Scientific) and 1% penicillin-streptomycin (Corning) and cultured at 37° C. in a humidified atmosphere with 5% CO₂. ADSCs were passaged at preconfluency using trypsin. The cells from passage 3 were used for the co-culture experiments.

In addition, NHDFs used in Example 1 were used.

### 1-2. PHH/ADSC Co-Culture and Production of Hepatocyte Construct

Primary human hepatocytes/ADSC co-culture and production of a hepatocyte construct were performed in the same manner as in Example 1 except that ADSCs were used in place of the above-mentioned NHDFs. However, ADSCs were seeded at 1×10⁵ cells/well.

### 2. Results

(a) to (d) of FIG. 9 show phase-contrast micrographs of PHH/ADSC co-culture cell sheets ((a) and (b)) and PHH/NHDF co-culture cell sheets ((c) and (d)) on day 28 after release. In all the PHH/ADSC co-culture cell sheets ((a) and (b)) and the PHH/NHDF co-culture cell sheets ((c) and (d)), accumulation of lipid droplets which is the characteristic of NASH was confirmed.

### Example 3

### 1. Experimental Materials & Methods

### 1-1. Cells

Primary mouse hepatocytes (PMH) were isolated from the liver of a mouse by a modified two-step collagenase perfusion method and purified by a 45% percoll density gradient centrifugation method. The viability of PMHs was 90%.

As NHDFs, NHDFs used in Example 1 were used.

1-2. Primary Mouse Hepatocytes (PMH)/NHDF Co-Culture and Production of Hepatocyte Construct

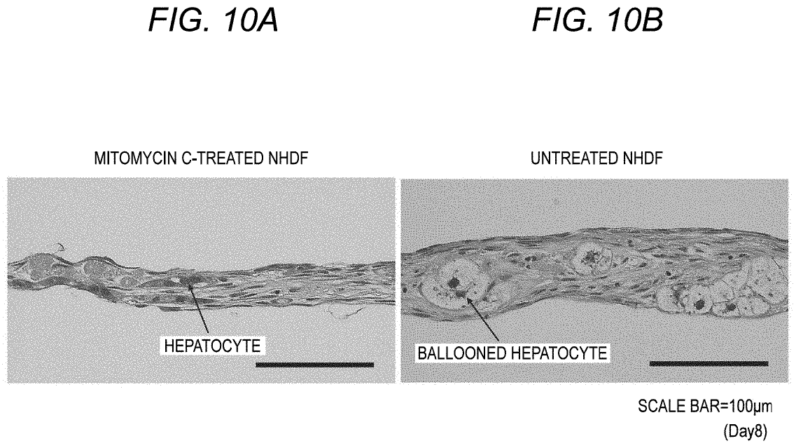

Primary mouse hepatocytes (PMH)/NHDF co-culture and production of a hepatocyte construct were performed in the same manner as in Example 1 except that PMHs were used in place of the above-mentioned PHHs. As the NHDFs, NHDFs treated with mitomycin C (6 μg/mL) for 2.5 hours before being co-cultured with PMHs or untreated NHDFs were used (FIGS. 10A and 10B).

### 2. Results

Mitomycin C has an effect of suppressing cell proliferation. It was revealed that hepatocellular ballooning is suppressed by the treatment with mitomycin C (see FIG. 10A).

### Example 4

### 1-1. Cells

Primary mouse hepatocytes (PMH) were obtained in the same manner as in Example 3.

As NHDFs, NHDFs used in Example 1 were used.

### 1-2. Primary Mouse Hepatocytes (PMH)/NHDF Co-Culture and Production of Hepatocyte Construct

Primary mouse hepatocytes (PMH)/NHDF co-culture and production of a hepatocyte construct were performed in the same manner as in Example 1 except that PMHs were used in place of the above-mentioned PHHs.

Obeticholic acid (10 μM) (Funakoshi, Cat. No. AG-CR1-3560-M025) developed as a therapeutic agent for NASH or metafomin (500 μNI) (Sigma, Cat. No. PHR1084-500MG) that is a therapeutic agent for type 2 diabetes was added to the above-mentioned hepatocyte construct on day 1, and culture was performed for 8 days.

### 2. Results

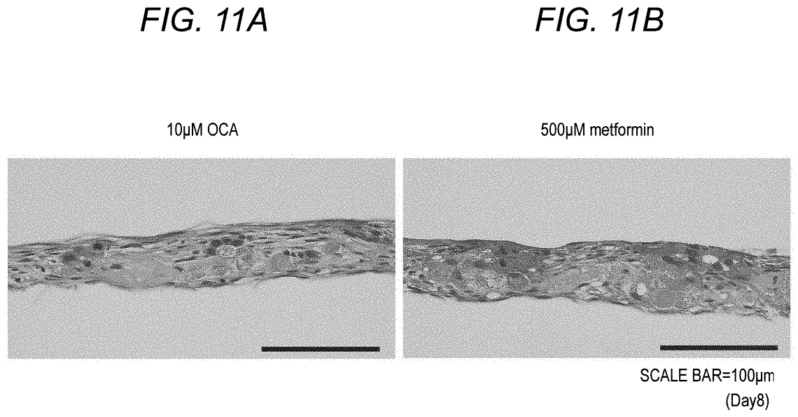

By the addition of obeticholic acid (OCA) and metformin, hepatocellular ballooning was suppressed (see FIGS. 11A and 11B).

### Example 5

### 1. Experimental Materials & Methods

### 1-1. Cells

As PHHs and NHDFs, PHHs and NHDFs used in Example 1 were used.

### 1-2. PHH/NHDF Co-Culture and Production of Hepatocyte Construct

Primary human hepatocytes (PHH)/NHDF co-culture and production of a hepatocyte construct were performed in the same manner as in Example 1 described above.

In the same manner as in Example 4, obeticholic acid (10 μM) (Funakoshi) or metafomin (500 μM) (Sigma) was added to the above-mentioned hepatocyte construct on day 1, and culture was performed for 4 to 11 days.

### 2. Results

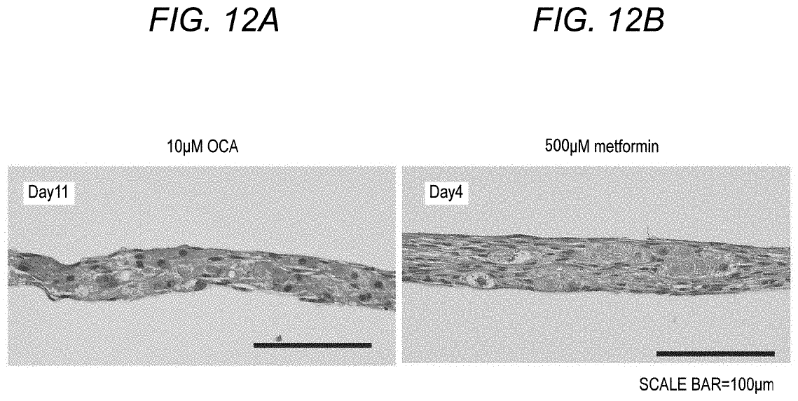

By the addition of each of obeticholic acid (OCA) and metformin, hepatocellular ballooning was suppressed (see FIGS. 12A and 12B).

### Example 6

### 1. Experimental Materials & Methods

### 1-1. Cells

As PHHs and NHDFs, PHHs and NHDFs used in Example 1 were used.

### 1-2. PHH/NHDF Co-Culture and Production of Hepatocyte Construct

Primary human hepatocytes (PHH)/NHDF co-culture and production of a hepatocyte construct were performed in the same manner as in Example 1 described above.

The above-mentioned hepatocyte construct was cultured for 4 days with a culture medium prepared under the condition of low glucose concentration (5.6 mM)+low insulin concentration (1 nM) or high glucose concentration (25 mM)+high insulin concentration (1 μM).

### 2. Results

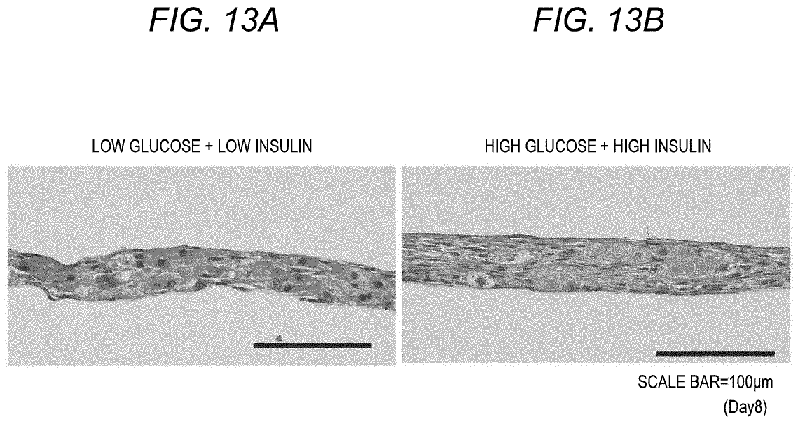

It was revealed that by the culture in the culture medium at low glucose concentration (5.6 mM)+low insulin concentration (1 nM), ballooning of the human hepatocytes was suppressed to some extent (see FIGS. 13A and 13B).

1. A hepatocyte construct comprising:
an aggregate containing hepatocytes and adherent cells that are non-hepatocytes,
wherein the hepatocytes include ballooned hepatocytes.

2. The hepatocyte construct according to claim 1, wherein the adherent cells are fibroblasts or mesenchymal stem cells.

3. The hepatocyte construct according to claim 1, wherein the adherent cells are primate fibroblasts.

4. The hepatocyte construct according to claim 1, wherein the aggregate is a cell sheet.

5. The hepatocyte construct according to claim 1, wherein sonic hedgehog (SHH) protein expression per cell is higher than that in normal hepatocytes.

6. The hepatocyte construct according to claim 1, wherein the hepatocytes contain a Mallory-Denk body.

7. The hepatocyte construct according to claim 1, wherein the hepatocytes are primary hepatocytes.

8. The hepatocyte construct according to claim 1, wherein the hepatocyte construct expresses α-SMA.

9. The hepatocyte construct according to claim 1, wherein the ratio of the hepatocytes to the adherent cells is from 10:1 to 1:10.

10. A method for producing a hepatocyte construct comprising:
(i) forming an aggregate by aggregating a cell group containing hepatocytes and adherent cells that are non-hepatocytes; and
(ii) culturing the aggregate.

11. The method according to claim 10, wherein the adherent cells are fibroblasts or mesenchymal stem cells.

12. The method according to claim 10, wherein the adherent cells are primate fibroblasts.

13. The method according to claim 10, wherein the hepatocytes are primary hepatocytes.

14. The method according to claim 10, wherein the forming comprises:
(i-1) seeding and culturing the hepatocytes on a first culture substrate;
(i-2) seeding and culturing the adherent cells on the first culture substrate on which the hepatocytes are seeded; and
(i-3) forming the aggregate by releasing the cell group containing the hepatocytes and the adherent cells from the first culture substrate.

15. The method according to claim 10, wherein the culturing is having the aggregate adhered onto a second culture substrate and culturing the aggregate.

16. The method according to claim 10, wherein in the forming, a ratio of the hepatocytes to the adherent cells is from 10:1 to 1:10.

17. A hepatocyte construct which is produced by the method according to claim 10.
